(19) United States
(12) Patent Application Publication
(10) Pub. No.: US 2018/0291066 A1
(43) Pub. Date: Oct. 11, 2018

(54) IN VIVO PRODUCTION OF LONG DOUBLE STRANDED RNA UTILIZING VLPS

(71) Applicant: APSE, LLC, St. Louis, MO (US)

(72) Inventors: Juan Pedro Humberto ARHANCET, St. Louis, MO (US); PATRICK D. MCLAUGHLIN, St. Louis, MO (US)

(73) Assignee: APSE, LLC, St. Louis, MO (US)

(21) Appl. No.: 15/570,711

(22) PCT Filed: May 9, 2016
(86) PCT No.: PCT/US16/31504

§ 371 Date: Oct. 30, 2017

Related U.S. Application Data
(60) Provisional application No. 62/160,329, filed on May 12, 2015.

(51) Int. Cl. C07K 14/005; C12N 7/00

(52) U.S. Cl. CPC C07K 14/005; C12N 2795/18142; C12N 2795/18123; C12N 7/00

(57) ABSTRACT

Methods and compositions for producing, isolating and purifying virus-like particles (VLPs) containing long sense and antisense RNA molecules, including RNA molecules predicted to fold into double-stranded regions longer than the inner diameter of the VLP capsids are disclosed.

### CROSS REFERENCE TO RELATED APPLICATIONS

This application claims priority to U.S. provisional application No. 62/160,329, filed May 12, 2015, the entire disclosure of which is hereby incorporated by reference.

### INCORPORATION OF SEQUENCE LISTING

The entire contents of a paper copy of the “Sequence Listing” and a computer readable form of the sequence listing on optical disk, containing the file named 103827-5005-WO-SEQ-ST25.txt, which is 1 KB in size and was created on May 6, 2016, are herein incorporated by reference.

### TECHNICAL FIELD

The present invention is directed to the production, isolation and purification of virus-like particles containing long double stranded RNA molecules.

### BACKGROUND OF THE INVENTION

Virus-like particles (VLPs) are derived in part from viruses through the expression of certain viral structural proteins which make up the viral capsid, but VLPs do not contain the viral genome and are non-infectious. Viral capsids are composed of at least one protein, several copies of which assemble to form the capsid. VLPs have been made containing RNA sequences with no substantial similarity to any virus, i.e. they encapsidate heterologous RNA.

Nucleic acids, including siRNA and miRNA, have for the most part been manufactured using chemical synthesis methods. These methods are generally complex and high cost because of the large number of steps needed and the complexity of the synthetic reactions. In addition, the synthetic reagents involved are costly and so economy of scale is not easily obtained by simply increasing batch size. Alternatively, in-vitro transcription methods have also been used to manufacture RNA. As with chemical synthesis methods, the reagents involved are also costly and likewise economy of scale is not easily obtained by simply increasing batch size.

Nucleic acids strands, including RNA strands, spontaneously adopt conformations that lower their free energy by complementary association with other strands with which they hybridize via non-covalent interactions. Watson-Crick base pairing occurs between C and G, and between A and U. Wobble base pairing occurs between G and U, and is less energetically favored than A and U base pairing, which is less energetically favored than C and G base pairing. Interactions between A and C, A and G, and U and C are non-pairing or mismatches. For every base not present in one of two otherwise perfectly complementary strands, a gap or a bulge may occur. From the point of view of the strand missing a base it is considered a gap, and from the point of view of the strand having a base in excess it is considered a bulge.

Inverted repeat sequences occurring within an RNA strand, for example sense and antisense sequences, spontaneously hybridize to form double stranded RNA (dsRNA) stems. The formation of such dsRNA regions may be quite rapid, especially relative to the time-scales required for complex biological operations such as capsid assembly. Such stems can be predicted using publicly available software, for example the “mfold” program [M. Zuker, Nucleic Acids Res. 31(13), 3406-15 (2003)] or the EINVERTED program. EINVERTED is part of the EMBOSS bioinformatics platform and is available from the European Molecular Biology Open Source Software Suite web site [Rice, P., Longden, I., and A. Bleasby, Trends in Genetics 16(6):276-77 (2000)]. Such dsRNA stems form double helical RNA structures. These double helices are approximately 0.28 nm/bp in length [Taylor, P., Rixon, F. and U. Desselberger, Virus Research 2:175-82 (1985)]. Stacking between two adjacent bases within the double helix is achieved because the planes containing each of them are essentially parallel. Base stacking represents the largest contribution to the stability of the double helix [Yakovchuk, P., Protozanova, E., and Frank-Kamenetskii, M. D., Nucleic Acids Research, 34: 564-74 (2006)]. RNA double helices behave essentially like rigid rods because base stacking stabilization is lowered by increasing the angle between stacking bases, which occurs if the double helix is bent.

Non-complementary bases in mismatches or bulges within the dsRNA stem, when present as a small fraction of the total number of bases, still stack with adjacent bases within the double helix [M. H. Bailor, A. M., et al., Current Opinion in Structural Biology, 21:296-305 (2011)]. Such non-complementary or mismatched bases do not significantly compromise the inherent rigidity of a long substantially perfectly complementary (SPC) dsRNA stem, which still behaves as an essentially rigid rod. The EINVERTED program can be used to obtain scores for SPC dsRNA stems. In contrast, RNA strands that don't form SPC dsRNA stems due to a large fraction of non-complementary bases adopt flexible, non-rigid conformations, which instead of behaving like a rigid rod, behave like a bicycle chain or a pearl necklace and may be randomly coiled or looped back upon themselves in an almost infinite variety of configurations.

A critical issue for packing RNAs with complex structure within capsids as contemplated by the present invention is the rate of formation of RNA structures relative to the overall capsid production and assembly of the VLPs. Transcription occurs quickly, in the range of 200-400 nucleotides per second, and the transcribed RNA strand can adopt secondary structures two to three orders of magnitude more quickly, on the microsecond time scale [C. Geary, P. Rothemund, E. Andersen, Science 345:799-804 (2014)]. On the other hand, capsid assembly around the folded RNA occurs at least two orders of magnitude more slowly than transcription [A. Borodavka, R. Tuma, and P. Stockley, PNAS 109:15769-774 (2012)].

RNA strands encapsidated within VLPs are constrained by the length of the longest SPC dsRNA stem that can rapidly form and still fit within the space available inside the capsid. For example, an MS2 capsid, having internal diameter of approximately 20 nm, cannot accommodate an SPC dsRNA stem much longer than about 70 base pairs (19.6 nm) [Kuzmanovic, D. A., et al., Structure 11 1339-48 (2003)]. It is therefore unsurprising that the literature has not previously described VLPs encapsidating heterologous RNA predicted to form SPC dsRNA longer than the internal diameter of the capsid. In fact, to our knowledge no encapsidated RNA predicted to form SPC dsRNA stems longer than 43 bps (˜13 nm) has previously been described in the literature [Pan et al., FEBS Journal 279(7):1198-208 (2012)]. Furthermore, no encapsidated RNA strand predicted to form a perfectly complementary dsRNA longer than 34 bp (˜10 nm) has been described in the literature [Klovins, J., J. Van Duin, and R. C. L. Olsthoorn. Nucleic Acids Research 25(21): 4201-08 (1997)]. Based on the existing literature and the present understanding of RNA structure there is no expectation by those skilled in the art that SPC sequences, for example inverted repeat sequences, or sense and antisense sequences longer than 70 nucleotides can be packaged within VLPs with inner diameters of approximately 20 nm such as those produced by bacteriophage Qβ or MS2. The rigid structure of such double stranded RNA is thought to be simply too long to fit within the inner confines of the capsid itself. However, such long sense and antisense RNA molecules have many useful properties and the ability to produce such molecules in vivo can significantly reduce production costs relative to in vitro synthesis. Packaging RNA within VLPs has been shown to provide an effective strategy for purifying in vivo synthesized RNA. Thus, methods for packaging long sense and antisense RNA strands within VLPs would constitute a significant advance in the art.

### SUMMARY OF THE INVENTION

The present invention relates to a method for producing long sense and antisense RNA strands in vivo. In several embodiments, the method comprises incubating an E. coli cell comprising an expression vector comprising a nucleic acid sequence encoding a capsid protein as well as a nucleic acid sequence encoding a long sense and antisense RNA strand operably linked to an expression control sequence under conditions suitable for the expression of the capsid protein and RNA sequence. The nucleic acid sequence encoding the long sense and antisense RNA strand preferentially also comprises a pac sequence allowing binding to the capsid protein to preferentially package the long sense and antisense RNA strand within the VLP. The E. coli cell comprising an expression vector comprising a nucleic acid sequence encoding a capsid protein and a long sense and antisense RNA strand sequence operably linked to an expression control sequence is grown under conditions suitable for the expression of the capsid protein and the long sense and antisense RNA strand. Within the E. coli cells in which they are expressed, the capsid proteins and the long sense and antisense RNA strands form VLPs in which the long sense and antisense RNA strands are packaged within the capsid formed by multiple capsid proteins. The E. coli cells are subsequently lysed and the VLPs recovered from the lysate. The substantially pure VLPs are then disrupted and the intact dsRNA isolated therefrom.

In one aspect, the nucleic acid sequence encoding a capsid protein comprises a leviviridae capsid protein gene operably linked to an inducible promoter sequence. Preferably the nucleic acid sequence encoding the capsid protein gene comprises the Qβ or MS2 capsid protein sequence. In some embodiments the nucleic acid sequence encoding the capsid protein operably linked to a promoter sequence is covalently linked to a nucleic acid sequence encoding the long sense and antisense RNA strand. In other embodiments the nucleic acid sequence encoding the capsid protein operably linked to a promoter sequence is not covalently linked to the long sense and antisense RNA strands. Preferably the expression of capsid protein and long sense and antisense RNA strands are inducible and the method comprises the steps of (a) growing the E. coli cell containing the vector encoding capsid protein and long sense and antisense RNA, and (b) inducing expression of capsid protein and long sense and antisense RNA strands, whether covalently linked or not.

In related aspects, the E. coli host cell containing a prophage, plasmid or other genetic element comprising T7 gene 1 or other DNA dependent RNA polymerases operably linked to an inducible promoter (e.g. a lac promoter) is transformed with an expression vector comprising a cognate promoter (e.g. a T7 promoter) operatively linked to the capsid protein coding sequence and a 19 base pac sequence followed by the desired heterologous long sense and antisense RNA strands sequence followed by an additional 19 base pac sequence. Expression of the capsid protein and the RNA sequence is induced by the addition of a suitable amount of inducer (e.g. Isopropyl β-D-1-thiogalactopyranoside (IPTG)) under conditions sufficient to produce large quantities of the desired capsid protein and long double-stranded RNA. The capsid protein and the long sense and antisense RNA strand spontaneously form VLPs within the E. coli host cell, and after a suitable period the host cells comprising the assembled VLPs are isolated and processed.

In related embodiments, fermentation of the host cells is conducted sufficient to yield at least about 0.1 mg, at least about 0.2 mg, at least about 0.5 mg, at least about 1.0 mg, at least about 2 mg, at least about 5 mg, at least about 10 mg, at least about 25 mg, at least about 50 mg, at least about 100 mg, at least about 0.5 g, at least about 1.0 g, at least about 2.5 g, at least about 5.0 g, at least about 4.5 g/L, at least about 5 g/L, at least about 10 g, at least about 25 g, at least about 50 g, at least about 100 g/L, at least about 250 g, at least about 500 g, or at least about 1.0 kg of substantially purified VLPs. In other embodiments, the amount of VLPs obtained is from about 0.1 mg to 10 mg, from about 10 mg to 100 mg, from about 100 mg to about 1.0 g, from about 1.0 g to about 10 g, from about 10 g to about 100 g, from about 100 g to about 1000 g.

In one aspect the present disclosure describes a VLP comprising a capsid enclosing at least one heterologous RNA strand predicted to form at least one SPC dsRNA stem at least 47 bp long. In another aspect the present disclosure describes a VLP comprising a capsid enclosing at least one heterologous RNA strand predicted to form at least one SPC dsRNA stem at least 71 bp long. In another aspect the present disclosure describes a VLP comprising a capsid enclosing at least one heterologous RNA strand predicted to form at least one SPC dsRNA stem at least 180 bp long. In yet another aspect the present disclosure describes a VLP comprising a capsid enclosing at least one heterologous RNA strand predicted to form at least one perfectly complementary dsRNA stem at least 35 bp long. VLPs according to the present disclosure may comprise a capsid which comprises a wild type capsid protein having at least 15%, at least 16%, at least 21%, at least 40%, at least 41%, at least 45%, at least 52%, at least 53%, at least 56%, at least 59% or at least 86% sequence identity with the amino acid sequence of wild type Enterobacteriophage MS2 capsid. The capsid may comprise wild type Enterobacteriophage MS2 capsid protein or Enterobacteriophage Qβ capsid protein.

The encapsidated heterologous RNA may be for example a siRNA, a miRNA or a shRNA. The present disclosure also encompasses a vector comprising any such nucleic acid constructs, and host cells comprising such a vector, as well as host cell stably transformed with such a vector. Host cells may be a bacterial cell, such as but not limited to an E. coli cell, a plant cell, a mammalian cell, an insect cell, a fungal cell or a yeast cell. A host cell may further be stably transfected with a second vector comprising a second nucleic acid sequence encoding a viral capsid. The second nucleic acid sequence may encode for example a viral protein encoding a viral capsid having at least 40% sequence identity with the amino acid sequence of wild type Enterobacteriophage MS2 capsid protein. A nucleic acid construct as described herein may also encode a wild type Enterobacteriophage MS2 capsid protein. The present disclosure also encompasses a plant or plant tissue transformed to contain a nucleic acid construct described herein and seed or progeny of such a plant or plant tissue, wherein the seed or progeny comprises the nucleic acid construct.

### DESCRIPTION OF THE DRAWINGS

FIG. 1 is a 1.25% TAE-agarose electrophoresis gel stained with Ethidium Bromide. Lane 1 and Lane 5 are molecular weight markers, Lane 2 is the ErkA specific 180 bp dsRNA product formed from 2 separate capsids, one that encodes the antisense strand and one that encodes the sense strand (as described in Example 3). Lane 3 is the sense strand alone, lane 4 is the antisense strand alone. Lane 6 is 171 micrograms of capsid containing both strands, lane 7 is 342 micrograms of capsid containing both strands, and lane 8 Is 684 micrograms of capsid containing both strands. Sample preparation is described in detail in Example 3.

### DETAILED DESCRIPTION OF THE INVENTION

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within the ranges. As used herein, the terms “about” and “approximately” when referring to a numerical value shall have their plain and ordinary meanings to one skilled in the pertinent art at issue. Also, the disclosure of ranges is intended as a continuous range including every value between the minimum and maximum values recited as well as any ranges that can be formed by such values. This includes ranges that can be formed that do or do not include a finite upper and/or lower boundary. This also includes ratios that are derivable by dividing a given disclosed numeral into another disclosed numeral. Accordingly, the skilled person will appreciate that many such ratios, ranges, and ranges of ratios can be unambiguously derived from the data and numbers presented herein and all represent various embodiments of the present invention.

A wide variety of conventional techniques and tools in chemistry, biochemistry, molecular biology, and immunology are employed and available for practicing the methods and compositions described herein, are within the capabilities of a person of ordinary skill in the art and well described in the literature. Such techniques and tools include those for generating and purifying VLPs including those with a wild type or a recombinant capsid together with the cargo molecule(s), and for transforming host organisms and expressing recombinant proteins and nucleic acids as described herein. See, e.g., Sambrook, et al., Molecular Cloning: A Laboratory Manual, 2nd Ed., Cold Spring Harbor Laboratory Press, Cold Spring Harbor, N.Y. (1989); and Ausubel, et al, Current Protocols in Molecular Biology, Greene Publ. Assoc., Wiley-Interscience, NY (1995). The disclosures in each of which are herein incorporated by reference.

Processes for preparing VLPs from genetically engineered bacterial host cells such as E. coli comprising expression systems are well known to those skilled in the art. For example, such systems are described in U.S. Patent Publication No. 20140302593 A1, the contents of which are incorporated herein by reference. In one aspect, the present methods relate to E. coli host cells comprising expression systems, the expression systems comprising nucleotide sequence encoding capsid proteins and a long sense and antisense RNA strand operably linked to an inducible promoter such that the capsid protein and the long sense and antisense RNA strand is expressed in the host cells when the promoter is induced. Introduction of the plasmid into the E. coli host cell can be accomplished by any of several standard molecular biology techniques such as those described in Davis, et al., Basic Methods in Molecular Biology, Elsevier Science Publishing Co., Inc., N.Y. (1986) and Sambrook, et al., Molecular Cloning: A Laboratory Manual, 2nd Ed., (1989), including, without limitation, calcium phosphate transfection, microinjection, electroporation, conjugation, infection and the like. Similarly, any system or vector suitable to maintain, propagate or express polynucleotides and/or express a polypeptide in a host may be used to practice the present invention. For example, the appropriate DNA sequence may be inserted into a vector such as a plasmid by standard techniques.

### A. Definitions

As used herein, the term “cargo molecule” refers to any oligonucleotide, polypeptide or peptide molecule, which is or may be enclosed by a capsid.

As used herein, the term “oligonucleotide” refers to a short polymer of at least two nucleotides. An oligonucleotide may be an oligodeoxyribonucleotide (DNA) or an oligoribonucleotide (RNA), and encompasses short RNA molecules such as but not limited to siRNA, shRNA, sshRNA, and miRNA.

As used herein, the term “peptide” refers to a polymeric molecule which minimally includes at least two amino acid monomers linked by peptide bond, and preferably has at least about 10, and more preferably at least about 20 amino acid monomers, and no more than about 60 amino acid monomers, preferably no more than about 50 amino acid monomers linked by peptide bonds. For example, the term encompasses polymers having about 10, about 20, about 30, about 40, about 50, or about 60 amino acid residues.

As used herein, the term “polypeptide” refers to a polymeric molecule including at least one chain of amino acid monomers linked by peptide bonds, wherein the chain includes at least about 70 amino acid residues, preferably at least about 80, more preferably at least about 90, and still more preferably at least about 100 amino acid residues. As used herein the term encompasses proteins, which may include one or more linked polypeptide chains, which may or may not be further bound to cofactors or other proteins. The term “protein” as used herein is used interchangeably with the term “polypeptide”.

As used herein, the term “variant” with reference to a molecule is a sequence that is substantially similar to the sequence of a native or wild type molecule. With respect to nucleotide sequences, variants include those sequences that may vary as to one or more bases, but because of the degeneracy of the genetic code, still encode the identical amino acid sequence of the native protein. Variants include naturally occurring alleles, and nucleotide sequences which are engineered using well-known techniques in molecular biology, such as for example site-directed mutagenesis, and which encode the native protein, as well as those that encode a polypeptide having amino acid substitutions. Generally, nucleotide sequence variants of the invention have at least 40%, at least 50%, at least 60%, at least 70% or at least 80% sequence identity to the native (endogenous) nucleotide sequence. The present disclosure also encompasses nucleotide sequence variants having at least about 85% sequence identity, at least about 90% sequence identity, at least about 85%, 86%, 87%, 88%, 89%, 90% 91%, 92%, 93%, 94%, 95%, 96%, 97%, 98%, 99%.

Sequence identity of amino acid sequences or nucleotide sequences, within defined regions of the molecule or across the full-length sequence, can be readily determined using conventional tools and methods known in the art and as described herein. For example, the degree of sequence identity of two amino acid sequences, or two nucleotide sequences, is readily determined using alignment tools such as the NCBI Basic Local Alignment Search Tool (BLAST) [Altschul et al., J. Mol. Biol. 215(3):403-10 (1990)], which are readily available from multiple online sources. Algorithms for optimal sequence alignment are well known and described in the art, including for example in Smith, T. F. and M. S. Waterman, Adv. Appl. Math. 2:482-9 (1981) and Pearson, W. R. and D. J. Lipman, Proc. Natl. Acad. Sci. (U.S.A.) 85: 2444-8 (1988). Algorithms for sequence analysis are also readily available in programs such as blastp, blastn, blastx, tblastn and tblastx. For the purposes of the present disclosure, two nucleotide sequences may be also considered “substantially identical” when they hybridize to each other under stringent conditions. Stringent conditions include high hybridization temperatures and low salt hybridization buffers which permit hybridization only between nucleic acid sequences that are highly similar. Stringent conditions are sequence-dependent and will be different in different circumstance, but typically include a temperature at least about 60° C., which is about 10° C. to about 15° C. lower than the thermal melting point (Tm) for the specific sequence at a defined ionic strength and pH. Salt concentration is typically about 0.02 molar at pH 7.

As used herein with respect to a given nucleotide sequence, the term “conservative variant” refers to a nucleotide sequence that encodes an identical or essentially identical amino acid sequence as that of a reference sequence. Due to the degeneracy of the genetic code, whereby almost always more than one codon may code for each amino acid, nucleotide sequences encoding very closely related proteins may not share a high level of sequence identity. Moreover, different organisms have preferred codons for many amino acids, and different organisms or even different strains of the same organism, e.g., E. coli strains, can have different preferred codons for the same amino acid. Thus, a first nucleotide acid sequence which encodes essentially the same polypeptide as a second nucleotide acid sequence is considered substantially identical to the second nucleotide sequence, even if they do not share a minimum percentage sequence identity, or would not hybridize to one another under stringent conditions. Additionally, it should be understood that with the limited exception of ATG, which is usually the sole codon for methionine, any sequence can be modified to yield a functionally identical molecule by standard techniques, and such modifications are encompassed by the present disclosure. As described herein below, the present disclosure specifically contemplates protein variants of a native protein, which have amino acid sequences having at least 15%, at least 16%, at least 21%, at least 40%, at least 41%, at least 52%, at least 53%, at least 56%, at least 59% or at least 86% sequence identity to a native nucleotide sequence.

The degree of sequence identity between two amino acid sequences may be determined using the BLASTp algorithm [Karlin, S. and S. F. Altschul, Proc. Natl. Acad. Sci. USA 87:2264-8 (1990). The percentage of sequence identity is determined by comparing two optimally aligned sequences over a comparison window, wherein the portion of the amino acid sequence in the comparison window may comprise additions or deletions (i.e. gaps) as compared to the reference sequence (which does not comprise additions or deletions) for optimal alignment of the two sequences. The percentage is calculated by determining the number of positions at which an identical amino acid occurs in both sequences to yield the number of matched positions, dividing the number of matched positions by the total number of positions in the window of comparison and multiplying the result by 100 to yield the percentage of sequence identity.

One of skill will recognize that polypeptides may be “substantially similar”, in that an amino acid may be substituted with a similar amino acid residue without affecting the function of the mature protein. Polypeptide sequences which are “substantially similar” share sequences as noted above, except that residue positions, which are not identical may have conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acid substitution groups include: valine-leucine-isoleucine, phenylalanine-tyrosine, lysine-arginine, alanine-valine, and asparagine-glutamine.

A nucleic acid encoding a peptide, polypeptide or protein may be obtained by screening selected cDNA or genomic libraries using a deduced amino acid sequence for a given protein. Conventional procedures using primer extension procedures, as described for example in Sambrook, et al, (1989) can be used to detect precursors and processing intermediates.

EINVERTED is publicly available software for identifying inverted repeats (stem-loops) in a nucleotide sequence. EINVERTED can be used to obtain various parametric scores for SPC dsRNA stems. Since EINVERTED doesn't take into account GU pairs it systematically underestimates the actual potential length of some SPC dsRNAs. To account for this, we have used the following protocol which we have named XEINVERTED, to obtain more accurate identification of the extent of SPC regions in an RNA strand.

First, EINVERTED identifies SPC sections of an RNA strand. Second, we evaluate immediately adjacent flanking sections for additional GU, AT and GC pairs. Third, we expand the identified SPC dsRNA stem to include such flanking sections. Fourth, we calculate the maximum possible score for the expanded SPC dsRNA stem. Fifth, we accept the expanded SPC ds RNA stem if the maximum possible score is higher than the EINVERTED score and XEINVERTED score for shorter SPC dsRNA stems, otherwise it is rejected. This ensures that more stable, but shorter, SPC dsRNA structures are properly recognized. The number of bases incorporated within an SPC region is referred to as the EINVERTED length or XEINVERTED length, depending on the method used to calculate that particular SPC region.

“SRP” is RNA capable of adopting a conformation comprising a substantially completely complementary stretch of dsRNA and is defined here as any RNA comprising 2 ssRNA sequences which, if occurring within the same RNA strand, has an EINVERTED length or an XEINVERTED length of at least 46 bp and an EINVERTED score or an XEINVERTED score greater than 50 when using the following calculational parameters:

Match score for AU & GC pairs=3

Match score for GU pairs=1

Mismatch score=−4

Gap penalty=5

Minimum score threshold=50

Maximum extent of repeats=3,000

Pan, et al., [Pan, et al., FEBS Journal 279(7): 1198-208 (2012)] describe encapsidation of sense and antisense RNA strands comprising SEQ ID NO.: 1 (P146 of Table 2, p. 1205 of Pan et al.), each containing a single SRP packaged into MS2. SEQ ID NO.: 1 represents an example of how a particular sequence can produce significantly different EINVERTED and XEINVERTED lengths and scores. Analysis of SEQ ID NO.: 1 with the parameters given above produces an EINVERTED length of 42 and EINVERTED score of 66. Applying the XEINVERTED protocol to SEQ ID NO.: 1, based on the same EINVERTED parameters produces an XEINVERTED length of 46 and XEINVERTED score of 85.

As described above, EINVERTED identified the two SPC strands within SEQ ID NO.: I as SEQ ID NO.:2, ACAUGAGGAUCACCCAUGUAGCUCUGAGAACUG AAUUCCAUGGGU on the sense strand, as well as the matching sequence SEQ ID NO.: 3, ACCUGUGAAAUUCAGUUCUUCAGCUACAUGAGGAUCACCCAUGU on the antisense strand.

XEINVERTED analysis reveals two additional GU matches, as well as an additional GC match, which results in expanding the two SPC strands within SEQ ID NO.: 1 as SEQ ID NO.: 4, CUACAUGAGGAUCACCCAUGUAGCUCUGAGAACU GAAUUCCAUGGGUU on the sense strand, as well as the matching sequence SEQ ID NO.: 5, GACCUGUGAAAUUCAGUUCUUCAGCUACAUGAGGAUCACC CAUGUGG on the antisense strand.

Thus, although EINVERTED provides a convenient tool for determining the basic parameters of SPC dsRNA, additional steps such as those described in the XEINVERTED protocol may reveal that a particular SPC dsRNA has significantly different parameters. This is important because the length of an SPC dsRNA is particularly relevant to determining whether it can be packaged within a VLP or not. In some cases EINVERTED may underestimate the length of an SPC dsRNA since it discriminates against non-canonical base pairing that is likely to occur in dsRNA formation.

“SRPs” is defined as more than one SRP.

### B. VLPS Enclosing RNA Capable of Adopting at Least One SRP Configuration

The methods and compositions described herein are the result in part of the appreciation that certain viral capsids can be prepared and/or used in novel manufacturing and purification methods to improve commercialization procedures for SRPs

The capsid may be a wild type capsid or a mutant capsid derived from a wild type capsid, provided that the capsid encloses a SRP.

The present disclosure encompasses a composition comprising: a plurality of virus-like particles each comprising a wild type viral capsid and at least one SRP in the wild type viral capsid. Subsequently the cargo molecules can be readily harvested from the capsids. Accordingly, such compositions are highly desirable for all applications where high purity and/or high production efficiency is required.

A capsid can self-assemble from capsid protein in the presence of nucleotide cargo, such as an oligoribonucleotide. In non-limiting example, a capsid as described herein may enclose a target heterologous RNA strand, such as for example a target heterologous RNA strand containing a total of between 1,800 and 2,248 ribonucleotides, including the 19-mer pac site from Enterobacteriophage MS2, such RNA strand transcribed from a plasmid separate from a plasmid coding for the capsid proteins, as described by Wei, et al., J. Clin. Microbiol. 46:1734-40 (2008), or transcribed from the same plasmid as the plasmid coding for the capsid proteins.

RNA interference (RNAi) is a phenomenon mediated by short RNA molecules such as siRNA molecules, which can be used for selective suppression of a target gene of interest, and has multiple applications in biotechnology and medicine. For example, short RNA molecules can be employed to target a specific gene of interest in an organism (the target host) to obtain a desirable phenotype. Longer dsRNA molecules, for example SRPs, can also be used for RNAi purposes. RNA molecules, including siRNA and SRPs, are however easily degraded by ubiquitous enzymes called RNAses. Capsids, such as those described herein, protect encapsidated RNA from enzymatic degradation.

VLPs containing RNA strands capable of forming SRPs, as described herein comprise any substantially perfectly complementary dsRNA longer than 42 bp identified by EINVERTED, or longer than 46 bp when identified using XEINVERTED, in which at least one of the strands comprises a sequence capable of hybridizing with RNA or DNA present in the target host at least 19 nt long. Such RNA or DNA present in the target host can be, or result from, target host genomic DNA or can be viral, bacterial or fungal sequences present in the target host before, during or after the SRPs are delivered to the target host. Examples of SRPs are pre-miRNAs, pre-siRNAs, miRNAs, certain hairpin-forming mRNAs, sections of dsRNA viruses and sections of replication intermediates of ssRNA viruses.

Such compositions are much simpler, less expensive and more reliably manufactured than current alternatives for RNA delivery.

VLPs as described herein may be assembled by any available method(s) which produces a VLP with an assembled capsid encapsidating one or more cargo molecule(s). For example, capsids and cargo molecules may be co-expressed in any expression system. Recombinant DNA encoding one or more capsid proteins, one or more cargo molecule(s) can be readily introduced into the host cells, e.g., bacterial cells, plant cells, yeast cells, fungal cells, and animal cells (including insect and mammalian cells) by transfection with one or more expression vectors by any procedure useful for introducing such a vector into a particular cell, and stably transfecting the cell to yield a cell which expresses the recombinant sequence(s).

The host cell is preferably of eukaryotic origin, e.g., plant, mammalian, insect, yeast or fungal sources, but non-eukaryotic host cells may also be used. Suitable expression systems include but are not limited to microorganisms such as bacteria {e.g. E. coli) transformed with recombinant bacteriophage DNA, plasmid DNA or cosmid DNA expression vectors containing the coding sequences for the VLP elements. In non-limiting example, E. coli is a suitable expression system for VLPs comprising the MS2 capsid protein.

The present disclosure expressly contemplates plant cells which have been transformed using a nucleic acid construct as described herein, and which expresses a capsid coat protein and cargo molecule. Means for transforming cells including plant cells and preparing transgenic cells are well known in the art. Vectors, plasmids, cosmids, YACs (yeast artificial chromosomes) and DNA segments can be used to transform cells and will as generally recognized include promoters, enhancers, and/or polylinkers. Transgenic cells specifically contemplated include transgenic plant cells including but not limited to cells obtained from corn, soybean, wheat, vegetables, grains, legumes, fruit trees, and so on, or any plant which would benefit from introduction of a VLP as described herein. Also contemplated are plants, plant tissue obtained from cells transformed as described herein, and the seed or progeny of the plant or plant tissue.

Expression of assembled VLPs can be obtained for example by constructing at least one expression vector including sequences encoding all elements of the VLP. Sometimes two vectors are used, a first vector which includes a sequence encoding the cargo molecule(s); and a second vector which includes a sequence encoding the capsid protein. In an exemplary process for generating exemplary VLPs comprising SRPs, one vector may be expressed in the host cell for generation of the VLP, as further detailed in the Examples. Methods and tools for constructing such expression vector containing the coding sequences and transcriptional and translational control sequences are well known in the art. Vector(s) once constructed are transferred to the host cells also using techniques well known in the art, and the cells then maintained under culture conditions for a time sufficient for expression and assembling of the VLPs to occur, all using conventional techniques. The present disclosure thus encompasses host cells containing any such vectors, and cells which have been transformed by such vectors, as well as cells containing the VLPs.

When the VLPs have been expressed and assembled in the host cell they may be isolated and purified using any method known in the art for virus purification. For example, the cells can be lysed using conventional cell lysis techniques and agents. VLPs in the cell lysate following hydrolysis can be removed and purified using conventional protein isolation techniques.

Purification of capsids, VLPs or proteins may also include methods generally known in the art. For example, following capsid expression and purification of capsids, VLPs or proteins may include for example at least one liquid-liquid extraction step, at least one fractional precipitation step, at least one ultrafiltration step, or at least one crystallization step. A liquid-liquid extraction may comprise for example use of an immiscible non-aqueous non-polar solvent, such as but not limited to benzene, toluene, hexane, heptane, octane, chloroform, dichloromethane, or carbon tetrachloride. Purifying may include at least one crystallization step.

Following purification, the capsid can be opened to obtain the cargo molecule, which may be a protein or polypeptide, a peptide, or a nucleic acid molecule as described herein. Capsids can be opened using any one of several possible procedures known in the art, including for example heating in an aqueous solution above 50° C.; repeated freeze-thawing; incubating with denaturing agents such as formamide; by incubating with one or more proteases; or by a combination of any of these procedures.

VLPs according to the present disclosure and as used in any of the methods and processes, thus encompass those comprising a capsid protein having at least 15%, 16%, 21%, 40%, 41%, 52%, 53%, 56%, 59% or at least 86% sequence identity with the amino acid sequence of wild type Enterobacteriophage MS2 capsid protein.

The specialized VLPs described herein can be used in research and development and in industrial manufacturing facilities to provide improved yields, since the purification processes used in both settings have the same matrix composition. Having such same composition mainly depends on using the same cell line in both research and development and manufacturing processes.

### C. Preferred Embodiments

The following are among the preferred embodiments of the invention.

In one embodiment, the invention is a method for producing VLPs containing heterologous RNA sequences capable of forming sense and antisense RNA strand regions longer than the internal diameter of the VLP capsid.

In another embodiment, the invention is a virus-like particle (VLP) comprising a capsid enclosing at least one heterologous cargo molecule and at least one packing sequence, wherein the heterologous cargo molecule comprises at least two RNA sequences one of which is the reverse complement of the other and each is longer than 35 nucleotides.

In yet another embodiment, the VLP comprising a capsid enclosing at least one heterologous cargo molecule and at least one packing sequence, wherein the heterologous cargo molecule comprises at least two RNA sequences one of which is the reverse complement of the other, the number of complementary nucleotides in each RNA strand is larger than the ratio of the internal diameter of the capsid expressed in nanometers and 0.28.

In yet another embodiment, the VLP comprises a capsid enclosing at least one heterologous cargo molecule and at least one packing sequence, wherein the heterologous cargo molecule comprises at least two RNA sequences one of which is the reverse complement of the other, the two RNA sequences are part of the same RNA strand.

In yet another embodiment, the VLP comprises a capsid enclosing at least one heterologous cargo molecule and at least one packing sequence, wherein the heterologous cargo molecule comprises at least two RNA sequences one of which is the reverse complement of the other, the two RNA sequences are part of RNA strands not covalently linked to each other.

In still another embodiment the invention is a virus-like particle (VLP) comprising a capsid enclosing at least one heterologous cargo molecule and at least one packing sequence, wherein the heterologous cargo molecule comprises at least one SRP longer than 46 base pairs and an EINVERTED score or XEINVERTED score greater than 50 when using the following calculation parameters: match score for AU and GC pairs=3, match score for GU pairs=1, mismatch score=−4 and gap penalty=5.

In yet another embodiment, the VLP comprising a capsid enclosing at least one heterologous cargo molecule and at least one packing sequence, wherein the heterologous cargo molecule comprises at least one SRP longer than 46 base pairs and an EINVERTED score or XEINVERTED score greater than 50 when using the following calculation parameters: match score for AU and GC pairs=3, match score for GU pairs=1, mismatch score=−4 and gap penalty=5, the number of base pairs in the SRP is larger than the ratio between the internal diameter of the capsid expressed in nanometers and 0.28.

In yet another embodiment, the VLP comprising a capsid enclosing at least one heterologous cargo molecule and at least one packing sequence, wherein the heterologous cargo molecule comprises at least one SRP longer than 46 base pairs and an EINVERTED score or XEINVERTED score greater than 50 when using the following calculation parameters: match score for AU and GC pairs=3, match score for GU pairs=1, mismatch score=−4 and gap penalty=5, the SRP is formed within RNA sequences contained the same RNA strand.

In yet another embodiment, the VLP comprising a capsid enclosing at least one heterologous cargo molecule and at least one packing sequence, wherein the heterologous cargo molecule comprises at least one SRP longer than 46 base pairs and an EINVERTED score or XEINVERTED score greater than 50 when using the following calculation parameters: match score for AU and GC pairs=3, match score for GU pairs=1, mismatch score=−4 and gap penalty=5, the SRP is formed within RNA sequences not covalently linked to each other.

In still another embodiment, the invention comprises a nucleic acid construct encoding a capsid protein and a transcribed sequence, the transcribed region further comprising two regions, wherein one region comprises the reverse complement of the other, such that the transcribed sequence is capable of forming a double stranded RNA of at least 35 base pairs, such transcribed sequence further comprising a pac sequence.

In yet another embodiment, the invention comprises a host cell comprising a nucleic acid construct encoding a capsid protein and a transcribed sequence, the transcribed region further comprising two regions, wherein one region comprises the reverse complement of the other, such that the transcribed sequence is capable of forming a double stranded RNA of at least 35 base pairs, such transcribed sequence further comprising a pac sequence.

In still another embodiment, the invention comprises a nucleic acid construct encoding a capsid protein and a transcribed sequence, the transcribed region further comprising two regions, wherein one region comprises the reverse complement of the other, such that the transcribed region comprises at least one SRP longer than 46 base pairs and an EINVERTED score or XEINVERTED score greater than 50 when using the following calculation parameters: match score for AU and GC pairs=3, match score for GU pairs=1, mismatch score=−4 and gap penalty=5.

In yet another embodiment, the invention comprises a host cell comprising a nucleic acid construct encoding a capsid protein and a transcribed sequence, the transcribed region further comprising two regions, wherein one region comprises the reverse complement of the other, such that the transcribed region comprises at least one SRP longer than 46 base pairs and an EINVERTED score or XEINVERTED score greater than 50 when using the following calculation parameters: match score for AU and GC pairs=3, match score for GU pairs=1, mismatch score=−4 and gap penalty=5.

### Example 1

### Production of VLPs Containing a Single Stranded RNA Comprising Sense and Antisense ErkA Sequence Flanked by PAC Sequences in a Single Capsid

Plasmid pAPSE10114 (SEQ ID NO.: 6) encodes a T7 promoter sequence upstream of the MS2 coat protein (SEQ ID NO: 7) followed by a multiple cloning site flanked by two distinct MS2 19-mer RNA hairpins to enhance packing of the target sequence (the target sequence site). The pac site found 5′ of the target sequence site has the wild-type sequence ACAUGAGGAUUACCCAUGU and the C-PAC site found 3′ of the target sequence site is a modified pac site with a higher affinity for binding the MS2 coat protein with the sequence ACAUGAGGAUCACCCAUGU. A T7 terminator is also encoded in the vector to stop transcription of the RNA. This construct is inserted into the SmaI restriction site of plasmid pBR322.

Cloning of sense and antisense 180 bp fragments of ErkA (SEQ ID NO: 8) from Drosophila melanogaster (GenBank Accession NM_001300706 positions 156-335) were performed by PCR amplification of the region of interest and subsequent ligation into pAPSE10114 (SEQ ID NO: 6) to form pAPSE10131 (SEQ ID NO: 9) which contains the ErkA sense and antisense strands connected by a 40 bp linker.

Chemically competent HTE115 (DE3) cells were transformed with pAPSE10131, and selected for ampicillin resistant transformants. For VLP production these transformants were grown at 37° C. in 100 mL LB medium containing ampicillin. When the culture density reached OD₆₀₀ 0.8, isopropyl β-D-thiogalactopyranoside (Gold Biotech, St. Louis, Mo.) was added to a final concentration of 1 mM. Cells were harvested 4 hours post-induction by centrifugation at 3,000 g at 4° C. for 10 minutes. Cells were stored at 4° C. until time of lysis.

### Example 2

Purification of VLPs by Enzymatic Treatment and Fractional Precipitation with Ammonium Sulfate

Purification of VLPs was conducted as follows. One half of the cell pellet obtained from Example 1 was resuspended in approximately 10 volumes of 20 mM Tris-HCl, pH 7.0, containing 10 mM NaCl and sonicated to lyse the cells. Cell debris was removed by centrifugation at 16,000 g. The sample was further processed by addition of Benzonase® Nuclease (Sigma Aldrich, St. Louis, Mo.) was added to a final concentration of 100 units per mL and incubated at 37° C. for two hours. Proteinase K was then added to final concentration of 150 micrograms per mL and incubated at 37° C. for an additional three hours. At this point the VLP sample was ready for fractional ammonium sulfate precipitation.

Fractional precipitation of VLPs was conducted as follows. A saturated ammonium sulfate solution was prepared by adding ammonium sulfate to water to a final concentration of 4.1 M. The saturated ammonium sulfate was added to the enzymatically treated VLPs to a final concentration of 186 mM (approximately a 1:22 dilution) and placed on ice for two hours. Unwanted precipitate was cleared from the lysate by centrifugation at 16,000 g. The sample was then subjected to a second precipitation by the addition of 155 mg of dry ammonium sulfate directly to each mL of cleared lysate. The sample was vortexed and incubated on ice for two hours. The precipitate was spun down at 16,000 g and the solid precipitate was kept and resuspended in one tenth the original volume of 20 mM Tris-HCl, pH 7.0, containing 10 mM NaCl.

### Example 3

### VLPs Produce Double-Stranded RNA Resistant to RNAse a Treatment

VLPs comprising MS2 capsids obtained from 100 mL of culture of Example 1 and purified as described in Example 2 were used to produce dsRNA as follows. The amount of capsid used for the experiment was quantified by measuring A₂₆₀ and by protein assay using a BCA Assay (Pierce® BCA Protein Assay Kit, Thermo Fisher Scientific, Rockford, Ill.). Protein concentration was determined to be 28.50 microgram/microliter. The A₂₆₀ value was 123. To test the ability for the RNA inside the capsid to form double-stranded RNA the following amounts of capsids were used: 171, 342, and 684 micrograms.

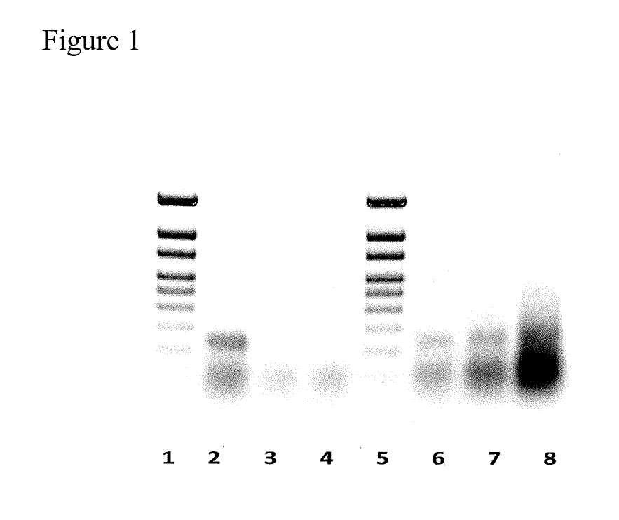

Incubation of 40 microliters of the capsids in 10 mM Tris-HCl, 300 mM NaCl, pH 7.0 at 75° C. for 5 minutes released the RNA into solution and which was subsequently allowed to cool to room temperature at a rate of 4° C. per minute. Once this was done the released RNA was treated with 0.400 nanograms of RNAse A (Ambion®, Life Technologies, Thermo Fisher Scientific, Rockford, Ill.) and incubated at 37° C. for 40 minutes. To deactivate the RNAse 0.001 units of Proteinase K from Tritirachium album was added and incubated at 37° C. for 40 minutes. The resultant RNA that was protected from RNAse A cleavage by virtue of being in double stranded form, was run on a 1.25% TAE-agarose gel stained with ethidium bromide. See FIG. 1. Lane 1 and Lane 5 are molecular weight markers, Lane 2 is the ErkA 180 bp dsRNA product formed from RNA isolated from the capsids of two separate VLPs, one that encapsidated the antisense strand and one that encapsidated the sense strand. Lane 3 is the sense strand alone, Lane 4 is the antisense strand alone. Lane 6 is RNA from 171 micrograms of VLPs containing a single RNA sequence including both sense and anti-sense sequences encapsidated within a single capsid. Lane 7 was loaded with 342 micrograms and Lane 8 684 micrograms of such VLPs.

### Example 4

### Production of VLP Preparations, One Comprising a Single Stranded RNA ErkA Sense Strand and Another the Complementary ErkA Antisense Strand, Each Flanked by PAC Sequences in Separate Capsids

A 180 bp fragment of ErkA (SEQ ID NO: 8) from Drosophila melanogaster (GenBank Accession NM_001300706 156-335) was amplified by PCR and subsequently ligated into the AsiSI and PmeI restriction sites of pAPSE10114 (SEQ ID NO: 6) to form pAPSE10120 (SEQ ID NO: 10) which contains the ErkA fragment in the sense orientation relative to the T7 promoter. A separate construct, pAPSE10121 (SEQ ID NO: 11) was similarly produced containing the ErkA fragment in the anti-sense orientation relative to the T7 promoter.

Chemically competent HTE115 (DE3) cells were separately transformed with pAPSE10120 and pAPSE10121 and individually selected for ampicillin resistant transformants. For capsid production each of these transformants were grown separately at 37° C. in 100 mL LB medium containing ampicillin. When the culture density reached OD₆₀₀ 0.8, isopropyl β-D-thiogalactopyranoside (Gold Biotech, St. Louis, Mo.) was added to a final concentration of 1 mM. Cells were harvested 4 hours post-induction by centrifugation at 3,000 g at 4° C. for 10 minutes. Cells were stored at 4° C. until time of lysis.

### Example 5

### VLPs of Example 4 are Capable of Producing Double-Stranded RNA Resistant to RNAse a Nuclease Treatment

VLPs comprising MS2 capsids obtained in Example 4 were purified as described in Example 2 and used to produce dsRNA by the following method. The amount of capsid used for the experiment was quantified by A₂₆₀ and by BCA Assay (Pierce® BCA Protein Assay Kit, Theinio Fisher Scientific, Rockford, Ill.). Based on these values equal amounts of VLPs from pAPSE10120 and pAPSE10121 were combined to a total of 30 micrograms of encapsidated RNA in a total reaction volume of 40 microliters.

As an alternative to Trizol extraction of the nucleic acid encapsulated in the VLPs, the following procedure was used to “heat-crack” the VLPs. Incubation of 40 microliters of the capsids in 10 mM Tris-HCl, 300 mM NaCl, pH 7.0 at 75° C. for 5 minutes released the RNA into solution, and then cooled to room temperature at a rate of 4° C. per minute. Once this was done the released RNA was treated with 0.400 nanograms of RNAse A (Ambion®, Life Technologies, Thermo Fischer Scientific, Rockford, Ill.) and incubated at 37° C. for 40 minutes. To deactivate the RNAse A, 0.001 units of Proteinase K from Tritirachium album was added and the solution incubated at 37° C. for an additional 40 minutes. The remaining RNA protected from RNAse A digestion was run on a 1.25% TAE-agarose gel stained with ethidium bromide. See FIG. 1. Lane 1 and lane 5 are molecular weight markers (Lambda 100 bp dsDNA Ladder, the markers are 100, 200, 300, 400, 500, 600, 800, 1000, and 1500 bp in size), Lane 2 is the ErkA 180 bp dsRNA product formed from 2 separate capsid sources, one containing the sense RNA strand (produced from pAPSE10120) and one containing the antisense RNA strand (produced from pAPSE10121). Lane 3 is the RNA sense sequence strand alone, Lane 4 is the RNA antisense sequence strand alone. Lane 6 is 171 microgram of capsids produced from pAPSE10131 containing RNA with an inverted repeat containing the sense and anti-sense sequences separated by a 40 base loop as described in Example 1. Lane 7 is 342 micrograms of RNA from capsids produced from pAPSE10131, and Lane 8 is 684 microgram of RNA produced from pAPSE10131 capsids.

The results clearly indicate that when the two VLPs, sense and antisense, are heat-cracked together the single-stranded RNA they contain may form a dsRNA product that corresponds to the 180 bp RNAse A resistant molecule. The same dsRNA product can also be formed from VLPs which encode the sense and antisense strands within a single RNA molecule encapsidated within a VLP. This is a surprising result since a 180 bp dsRNA is predicted to have a length of about 40 nanometers, which is too long to fit within the approximately 20 nanometer diameter interior cavity of MS2. This indicates that the RNA is incorporated into the capsid in a manner which allows for packing of SRPs of significantly larger size in a single capsid.

Those skilled in the art will understand that plasmids similar to pAPSE10120, pAPSE10121 and pAPSE10131 may easily be constructed to produce sense and anti-sense RNA products of any particular sequence or length by substituting different DNA sequences cloned in place of the ErkA specific sequences described in this Example. Furthermore, those of skill in the art will also understand that producing RNA strands entirely lacking pac site sequences can be accomplished by deleting, or not incorporating, such sequences from expression plasmid pAPSE10114. For example, plasmid pAPSE10114 lacking the sequences between 908-926 and 11199-1137 provides a suitable expression platform to produce RNA molecules lacking pac site sequences.

### Example 6

In-Vitro Production of MS2 Capsids Self-Assembled in Presence of 30 bp dsRNA Flanked by MS2 PAC Sequences

Thirty bp dsRNA flanked by MS2 pac sequences can be produced in-vitro by simultaneous transcription of 30 nucleotides sense and antisense strands, each having an MS2 pac site at the 3′ end. MS2 coat protein is prepared and MS2 capsids are then allowed to spontaneously assemble into VLPs in the solution containing the annealed RNA strands as described in Ashley, et al., [Ashley, et al., ACSNano 6(3):2174-88 (2012)].

The in-vitro assembled MS2 VLPs are purified by treatment with 100 units of Benzonase® Nuclease (Sigma Aldrich, St. Louis, Mo.) and incubation at 37° C. for two hours. One hundred and fifty micrograms of Proteinase K is added and incubated at 37° C. for an additional three hours. At this point the VLPs may be purified by ammonium sulfate precipitation as described in Example 2.

The purified MS2 capsids are incubated in 10 mM Tris-HCl, 300 mM NaCl, pH 7.0 at 75° C. for 5 minutes to release the RNA into solution and allowed to cool to room temperature at a rate of 4° C. per minute. Once cooled, the released RNA is treated with 0.400 nanograms of RNAse A (Ambion®, Life Technologies, Thermo Fischer Scientific, Rockford, Ill.) and incubated at 37° C. for 40 minutes. To deactivate the RNAse A 0.001 units of Proteinase K from Tritirachium album is added and incubated at 37° C. for 40 minutes. The resultant RNA protected from RNAse A cleavage by virtue of being in a double stranded form is electrophoresed on a 1.25% TAE-agarose gel stained with ethidium bromide. A strong band of about 30 bp dsRNA indicates that double-stranded RNA was successfully packaged and purified.

### Example 7

In-Vitro Production of MS2 Capsids Self-Assembled in Presence of 180 bp dsRNA Flanked by MS2 PAC Sequences

One hundred and eighty bp dsRNA flanked by MS2 pac sequences can be produced in-vitro by simultaneous transcription of 180 nucleotides sense and antisense strands, each having an MS2 pac site at the 3′ end. MS2 coat protein is prepared and MS2 capsids are then allowed to spontaneously assemble into VLPs in the solution containing the annealed RNA strands as described in Example 6.

The in-vitro assembled MS2 VLPs are purified by treatment with 100 units of Benzonase® Nuclease (Sigma Aldrich, St. Louis, Mo.) and incubation at 37° C. for two hours. One hundred and fifty micrograms of Proteinase K is added and incubated at 37° C. for an additional three hours. At this point the VLPs may be purified by ammonium sulfate precipitation as described in Example 2.

The purified MS2 capsids are incubated in 10 mM Tris-HCl, 300 mM NaCl, pH 7.0 at 75° C. for 5 minutes to release the RNA into solution and allowed to cool to room temperature at a rate of 4° C. per minute. Once this is done the released RNA is treated with 0.400 nanograms of RNAse A (Ambion®, Life Technologies, Thermo Fischer, Rockford, Ill.) and incubated at 37° C. for 40 minutes. To deactivate the RNAse A, 0.001 units of Proteinase K from Tritirachium album is added and the solution incubated at 37° C. for an additional 40 minutes. The resultant RNA protected from RNAse A cleavage by virtue of being in a double stranded form is electrophoresed on a 1.25% TAE-agarose gel stained with ethidium bromide. Absence of a band of about 180 bp dsRNA indicates that double-stranded RNA was not successfully packaged and purified.

These results indicate that the methods described here are suitable for packaging and purifying long sense and antisense RNA strand molecules of a type not previously reported in the literature. Such methods facilitate inexpensive large scale RNA synthesis of complex RNA molecules with long stretches of complementary sequence capable of forming double-stranded structures.

What is claimed is:

1. A virus-like particle (VLP) comprising a capsid enclosing at least one heterologous cargo molecule and at least one packing sequence, wherein the heterologous cargo molecule comprises at least two RNA sequences one of which is the reverse complement of the other and each is longer than 35 nucleotides.

2. The VLP of claim 1 in which the number of complementary nucleotides in each RNA strand is larger than the ratio of the internal diameter of the capsid expressed in nanometers and 0.28.

3. The VLP of claim 1 wherein the two RNA sequences are part of the same RNA strand.

4. The VLP of claim 1 wherein the two RNA sequences are part of RNA strands not covalently linked to each other.

5. A virus-like particle (VLP) comprising a capsid enclosing at least one heterologous cargo molecule and at least one packing sequence, wherein the heterologous cargo molecule comprises at least one substantially completely complementary sequence of dsRNA (SRP) longer than 46 base pairs and an EINVERTED score or XEINVERTED score greater than 50 when using the calculation parameters: match score for AU and GC pairs=3, match score for GU pairs=1, mismatch score=−4 and gap penalty=5.

6. The VLP of claim 5 in which the number of base pairs in the SRP is larger than the ratio between the internal diameter of the capsid expressed in nanometers and 0.28.

7. The VLP of claim 5 wherein the SRP is formed within RNA sequences contained the same RNA strand.

8. The VLP of claim 5 wherein the SRP is formed within RNA sequences not covalently linked to each other.

9. A nucleic acid construct encoding a capsid protein and a transcribed sequence, the transcribed region further comprising two regions, wherein one region comprises the reverse complement of the other, such that the transcribed sequence is capable of forming a double stranded RNA of at least 35 base pairs, such transcribed sequence further comprising a pac sequence.

10. A host cell comprising the nucleic acid construct of claim 9.

11. A nucleic acid construct encoding a capsid protein and a transcribed sequence, the transcribed region further comprising two regions, wherein one region comprises the reverse complement of the other, such that the transcribed region comprises at least one SRP longer than 46 base pairs and an EINVERTED score or XEINVERTED score greater than 50 when using the calculation parameters: match score for AU and GC pairs=3, match score for GU pairs=1, mismatch score=−4 and gap penalty=5.

12. A host cell comprising the nucleic acid construct of claim 11.


SEQUENCE LISTING (12 sequences)

SEQ ID NO: 1 - RNA, 115 nt, Artificial Sequence
  RNA sequence P146 of Pan et al., (Pan et al., FEBS Journal 279(7) 1198-208 (2012), p. 1205, Table 2)
agaucuacaugaggaucacccauguagcucugagaacugaauuccauggguuauaucaaugucagaccugugaaauucaguucuucagcuacaugaggaucacccaugugguacc

SEQ ID NO: 2 - RNA, 45 nt, Artificial Sequence
  Sense strand portion of the P146 SPC dsRNA of SEQ ID NO. 1 identified by EINVERTED using the EINVERTED parameters described in paragraph [0043].
  primer_bind (1)..(45)
acaugaggaucacccauguagcucugagaacugaauuccaugggu

SEQ ID NO: 3 - RNA, 45 nt, Artificial Sequence; identical to SEQ ID NO: 2
  Antisense strand portion of the P146 SPC dsRNA of SEQ ID NO. 1 identified by EINVERTED using the EINVERTED parameters described in paragraph [0043].
  primer_bind (1)..(45)

SEQ ID NO: 4 - RNA, 48 nt, Artificial Sequence
  Sense strand portion of the P146 SPC dsRNA of SEQ ID NO. 1 identified by XEINVERTED using the EINVERTED parameters described in paragraph [0043].
  primer_bind (1)..(48)
cuacaugaggaucacccauguagcucugagaacugaauuccauggguu

SEQ ID NO: 5 - RNA, 47 nt, Artificial Sequence
  Antisense strand portion of the P146 SPC dsRNA of SEQ ID NO. 1 identified by XEINVERTED using the EINVERTED parameters described in paragraph [0043].
  primer_bind (1)..(47)
gaccugugaaauucaguucuucagcuacaugaggaucacccaugugg

SEQ ID NO: 6 - DNA, 5851 bp, Artificial Sequence
  DNA sequence of plasmid pAPSE10114.
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcccggtacccagatctcgatcccgcgaaattaatacgactcactatagggagaccacaacggtttccctctagatcacaagtttgtacaaaaaagcaggctaagaaggagatatacatatggcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggtgtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctactaatagacgccggccattcaaacatgaggattacccatgtatttaaatacccatgtccaggcgcgctccgcgatcgcacgcggacaactactacagggtttaaacctttcggattataacatcacatctaggcgcgcctgacgatcaaccataccagacggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatccgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 7 - DNA, 396 bp, Bacteriophage MS2
  CDS (1)..(390)
atggcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggtgtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctactaatag

SEQ ID NO: 8 - protein, 130 aa, Bacteriophage MS2
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 9 - DNA, 180 bp, Drosophila melanogaster
  misc_feature (1)..(180)
agttcctcaatctaatgctgaagttataaggggacaaatatttgaagttggtcctaggtatattaaactcgcctatataggtgaaggagcttatggcatggttgtgtctgcggatgacacgctaacaaaccaaagagttgcaataaaaaaaatatcgccctttgaacaccaaacttattg

SEQ ID NO: 10 - DNA, 6171 bp, Artificial Sequence
  DNA sequence of plasmid pAPSE10131.
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcccggtacccagatctcgatcccgcgaaattaatacgactcactatagggagaccacaacggtttccctctagatcacaagtttgtacaaaaaagcaggctaagaaggagatatacatatggcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggtgtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctactaatagacgccggccattcaaacatgaggattacccatgtatttaaatacccatgtccaggcgcgctccgcgatcgcagttcctcaatctaatgctgaagttataaggggacaaatatttgaagttggtcctaggtatattaaactcgcctatataggtgaaggagcttatggcatggttgtgtctgcggatgacacgctaacaaaccaaagagttgcaataaaaaaaatatcgccctttgaacaccaaacttattggtttaaacctttcggattataacatcacatctaggcgcgcccaataagtttggtgttcaaagggcgatattttttttattgcaactctttggtttgttagcgtgtcatccgcagacacaaccatgccataagctccttcacctatataggcgagtttaatatacctaggaccaacttcaaatatttgtccccttataacttcagcattagattgaggaactcggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatccgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 11 - DNA, 6011 bp, Artificial Sequence
  DNA sequence of plasmid pAPSE10120
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcccggtacccagatctcgatcccgcgaaattaatacgactcactatagggagaccacaacggtttccctctagatcacaagtttgtacaaaaaagcaggctaagaaggagatatacatatggcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggtgtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctactaatagacgccggccattcaaacatgaggattacccatgtatttaaatacccatgtccaggcgcgctccgcgatcgcagttcctcaatctaatgctgaagttataaggggacaaatatttgaagttggtcctaggtatattaaactcgcctatataggtgaaggagcttatggcatggttgtgtctgcggatgacacgctaacaaaccaaagagttgcaataaaaaaaatatcgccctttgaacaccaaacttattggtttaaacctttcggattataacatcacatctaggcgcgcctgacgatcaaccataccagacggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatccgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 12 - DNA, 6011 bp, Artificial Sequence
  DNA sequence of plasmid pAPSE10121
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcccggtacccagatctcgatcccgcgaaattaatacgactcactatagggagaccacaacggtttccctctagatcacaagtttgtacaaaaaagcaggctaagaaggagatatacatatggcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggtgtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctactaatagacgccggccattcaaacatgaggattacccatgtatttaaatacccatgtccaggcgcgctccgcgatcgccaataagtttggtgttcaaagggcgatattttttttattgcaactctttggtttgttagcgtgtcatccgcagacacaaccatgccataagctccttcacctatataggcgagtttaatatacctaggaccaacttcaaatatttgtccccttataacttcagcattagattgaggaactgtttaaacctttcggattataacatcacatctaggcgcgcctgacgatcaaccataccagacggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatccgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa